(19) United States
(12) Patent Application Publication
(10) Pub. No.: US 2007/0082056 A1
(43) Pub. Date: Apr. 12, 2007

(54) ANTIBIOTIC PEPTIDE PRODUCT

(76) Inventor: James W. Sawhill, Roseville, CA (US)

Correspondence Address: RICHARD D. FUERLE, 1711 W. RIVER RD., GRAND ISLAND, NY, 14072, US

(21) Appl. No.: 11/163,275

(22) Filed: Oct. 12, 2005

(51) Int. Cl. A61K 35/20; C12P 21/06

(52) U.S. Cl. 424/535; 435/68.1

(57) ABSTRACT

An antibiotic peptide is made by hydrolyzing milk protein. The hydrolysis is performed with a fungal protease in a single step at a protein concentration of 21 to 35 wt % to a DH of at least about 12%. The resulting antibiotic peptide has at least a log 2 kill of bacteria. It may be coated onto plants or animals or used in foods, medicines, ointment, or pastes.

### BACKGROUND OF THE INVENTION

This invention relates to an antibiotic peptide made by hydrolyzing milk proteins. In particular, it relates to an antibiotic peptide that is made from whey protein concentrate (WPC) at a concentration of at least 21 wt % in a single hydrolyzation step to a degree of hydrolyzation (DH) of at least about 12%.

The normal enzymatic production of peptides from proteins, including from whey proteins, produces very offensive-tasting peptide products. A list of the offending enzymes appears in Sawhill U.S. Pat. No. 6,787,158, herein incorporated by reference and referred to as “the Sawhill patent.” In that patent, two conditions were found to be essential to produce a desirable, good-tasting product that was not antigenic (i.e., it did not cause an adverse reaction in people sensitive to milk proteins). The first condition was a high degree of DH, which was paramount for the elimination of antigenicity. The second condition was that the enzymatic hydrolysis had to be performed with one of three proteases. The process of the Sawhill patent requires a full 48 hour digestion and two additions of enzyme to make non-antigentic peptides that have the desired non-antigencity. The second condition required a dilute solution of the substrate protein (about 5 to 20 wt %) to insure no steric hindrance. Otherwise, the DH percentage would be too low to produce a non-antigentic final product.

### SUMMARY OF THE INVENTION

The process of this invention uses a single step fungal protease to digest milk protein to produce an antibiotic peptide that is active against a broad spectra of bacteria, including coliforms, as well as yeast, mold, and fungus. The amount of kill is high enough so that the finished product is considered to be an antibiotic for bacteria, yeast, molds, and fungi. The antibiotic peptide also has a good flavor so that it can be used in foods without creating an undesirable taste.

While in the Sawhill patent the DH was high (about 22 to 40%), in the process of this invention, the DH is lower (about 12 to 25%). Also, there is no limit on the level of dissolved WPC in the substrate and 25%, and even 30% concentrations are acceptable; steric hindrance is not a problem. Two enzymatic steps were needed in the Sawhill patent, but in this invention only a single enzymatic step is required. The Sawhill patent required a 48 hour digestion, but in the process of this invention, only 24 hours are needed. And, while in the Sawhill patent the bacterial counts ended up at about 10,000 tpc/gm, in the process of this invention the counts are less than 100 tpc/gm. As a result, much higher concentrations of substrate WPC can be used to make acceptable peptides with antibiotic properties. Finally, the mix of peptides in the product of this invention is not the same as the mix of peptides in the product of the Sawhill patent.

Unexpectedly, peptides made according to the process of this invention have antibiotic activity not only against bacteria, but also against yeasts, molds, and fungus. Until now, the only three compounds that have both antibiotic activity against both bacteria and fungi were propionate, benzoate, and sorbate. The three types of compounds may be in either the acid form or in the salt form. They are restricted to a 0.1% concentration to be GRAS (generally regarded as safe). Peptides produced according to the process of this invention have an unlimited GRAS percentage.

### DESCRIPTION OF THE PREFERRED EMBODIMENTS

The process of this invention produces an antibiotic peptide, where “antibiotic” means that the peptide has at least a log 2 kill of bacteria. That is, an adequate amount of the peptide will kill 99% of the bacteria that it is in contact with within 4 hours. The antibiotic peptide of this invention will also kill yeasts, molds, and fungi to at least a log 2 kill, though additional digestion time may be required to make antibiotic yeasts, molds, and fungi. In contrast, the peptides of the Sawhill patent are non-antigenic (i.e., they do not cause an adverse reaction in a person, such as pain, swelling up, or difficulty in breathing, when placed in a food or a medicine), and these peptides are also antibiotic in their reaction against bacteria. This antiobiotic activity is less intense than the antiobiotic activity of a 15 formula. The log kill on bacteria is 1.5 to 1.8, whereas in a 15 formula the log kill is over 2. The log kill of the coliforms in a 15 formula is over 3 and as high as a log 4

Any type of milk protein may be used as the starting material in this invention. This includes all of the different whey proteins and casein in the milk portion of proteins. Casein from milk by pH 5 precipitation followed by dissolving the sodium caseinate in water may be used. Whey proteins can come from several sources but they all come from whole whey as a byproduct of the manufacture of cheese. Acid whey is another whey protein source that may be used, although it does not come from cheese production, but is a byproduct from making cottage cheese.

It is best to start the enzymatic digestion with either a soluble protein solution or a homogenized protein solution of an animal or vegetable tissue. All the milk proteins, such as casein and the whey proteins, can be used. The whey proteins can be separated into their component proteins and each will be easily digested by the procedure of this invention. It is best to use a protein fraction that has gone through a minimum of heating and agitation in order to ensure that the protein is in good shape when it is processed through the membranes in order to make a high quality WPC. Casein, which is made from milk, is not preferred because casein is more expensive and has a less desirable amino acid profile and soy protein has lower nutritional values. The preferred protein is whey, the liquid that remains after the removal of the cheese curds, the remaining liquid by-product, on an anhydrous basis is typically about 14% protein, about 70% lactose, and the remainder milk minerals.

Whey is obtained as a by-product of the manufacturing of cheese; it is that portion of the milk that is not in the cheese. Whey is a solution that leaves the cheese vats after the cheese solids (about 10%) have been removed. After the removal of the cheese, whey is about 6 to about 8 wt % solids, but it can be concentrated under vacuum to about 40 wt % dissolved solids. To prepare a dried product from whey, water is evaporated under vacuum and the product is dried. The dried product has a variety of uses, including as cattle feed and as a source of lactose, which is used in the manufacturing pills.

Whey protein concentrate (WPC) is the high protein fraction of the whey. It is made from the whole whey solution by passing the solution through a membrane that retains larger molecules but permits smaller molecules to pass through. The lactose and phosphates pass through the membrane, but the proteins don't. The retained protein fraction from the membrane step is concentrated to about 40 wt % and is spray dried to form WPC. The WPC is sold according to the percentage of protein in it, i.e., WPC 65 is 65 wt % protein, WPC 80 is 80 wt % protein, etc., with the remainder being the other solid components in the milk, such as lactose, butterfat, and ash. While WPC with any percentage of protein in it can be used, a WPC having about 65 to about 80 wt % protein is preferred because at lower percentages of protein and higher percentages of lactose, it is difficult to maintain a white color during the enzymatic digestion step. There are some WPC 100's available, but they are more expensive due to the difficulties of removing the remaining 20 wt % contaminants present in the WPC 80 preparations. There are two main protein fractions in the protein mixtures in whey. The smallest in size is about 1 80 amino acids long and the large one is about 350 amino acids long.

A protease (also, “proteinase”), an enzyme that hydrolyzes protein, is used to catalyze the hydrolyzation of the protein. Enzymatic hydrolysis is the process of splitting the peptide bond of a protein in an aqueous solution. When a peptide bond is broken, one alpha amino group and one carboxyl acid group are formed. The protease used greatly affects the quality of the resulting peptide product. Proteases from bacteria should not be used as the digested WPC has an unacceptable bitter taste when those proteases are used. Proteases from plants and animals should also not be used as they produce an off-flavor in the digested WPC. That is, the flavor may not be bad-tasting, but it is a different flavor than the WPC had. After a great deal of experimentation, it has been determined that proteases prepared from fungi (“fungal proteases”) are effective and do not produce a peptide product that has a bitter taste or an off-flavor. Any fungal protease may be used. Examples of suitable fungal proteases include “Flavourzyme,” (sold by Novo), made from subtiliasin Carlsburg, and “Proteinase 400,000,” “Proteinase 500,000,” “Fungal 500,000,” and “Fungal Concentrate,” (sold by Genencor), made from Aspergillus orzae. The preferred fungal proteases are “Flavourzyme” and “Fungal 500,000” because they are more effective.

Each fungal protease is actually a mixture of various enzymes that are believed to be structurally closely related, though the actual structure of each enzyme in the mixture and its proportion in the mixture have not yet been determined. Because the fungal protease is a mixture, the digestion of a protein will go in several steps, cleaving at different junctions. The protein will be cut first into several fairly long peptides, then into smaller pieces. About 50% of the product will be free amino acid and 2 to 4 amino acid chains and the rest will be longer peptides.

The DH gives the percentage of the peptide linkages that have been broken. For example, a DH of 25% means that 25% of the peptide linkages have been broken, forming basic amine groups and acidic carboxylic acid groups.

An aqueous solution of the protein is hydrolyzed using the fungal protease. The protein is used at about 21 to about 35 wt % (based on composition weight). More protein will make the solution thick and difficult to stir during the enzymatic digestion and may lead to bacterial spoilage before the peptide becomes antibiotic. Less protein will increase the amount of enzyme used in the production, and therefore its cost. Also larger tanks will be needed, which will add to the cost; the preferred amount of protein is about 25 to about 33 wt %. The protein is mixed with about 0.01% to about 1 wt % (based on total composition weight) of a fungal protease. More fungal protease may not produce a proportional increase in DH and less fungal protease may not increase the DH to the required percentage. The preferred amount of fungal protease is about 0.05 to about 0.3 wt %.

A pH range of about 3 to about 5.5 is necessary during the hydrolysis in order to obtain peptides that are antibiotic. At lower than about 3 the antibiotic activity begins to lessen and at a pH above about 5.5 the antibiotic qualities diminish. The preferred pH range is about 3.5 to about 4.5. The pH is controlled somewhat by the buffering effect of the free carboxylic acid and amine groups that are formed when the peptide is hydrolyzed. That buffering effect is very strong and holds the pH of the system at about 4.2, which is approximately the pH that maximizes antibiotic properties of the peptide.

For best results, the protein to be hydrolyzed should be at a temperature of about 80 to about 120° F. At lower temperatures the reaction slows down and considerably more time is required to achieve sufficient digestion. Also, there is the real possibility of bacterial spoilage before the DH reaches levels adequate to insure that the product is antibiotic. This digestion is a first order chemical reaction, so it is very temperature sensitive. For every 18 degrees decrease in temperature during the enzymatic digestion the reaction rate falls by half. The preferred temperature range is about 105 to about 110° F. The pH is allowed to drift in an 80% protein WPC during the enzymatic digestion, but when a 34%.protein WPC is used it is adjusted to 5 before the enzymatic digestion is started. DH is followed and the enzymatic step is complete, as far as the production of antibiotic properties is concerned, when the DH is over about 12%, and preferably over about 15%.

The mixture is stirred and is monitored by high pressure liquid chromatography (HPLC) to determine the percentage of the protein that has been hydrolyzed. The DH at the end of the digestion step may vary from about 12 to about 25%. The DH is obtained by titration, measuring the amount of base needed to raise the pH to 8. Preferably, about 15 to about 20 wt % of the protein is hydrolyzed in the enzymatic hydrolyzation. The enzymatic hydrolyzation should be permitted to continue until the rate of hydrolyzation falls significantly. This can be determined by monitoring the DH, but it is usually convenient to simply run the hydrolyzation for 24 hours even though in many cases a sufficiently high DH may be achieved in less time. The time for the digestion can vary as needed, but a DH within the required range can usually be obtained in less than 24 hours and certainly in less than 48 hours.

The resulting antibiotic peptide, with a DH of 15, is a mixture of peptides with an average amino acid length of 7 to 8 amino acid groups. In contrast, the product of the Sawhill patent, with a DH of 25, has an average peptide length of 4 amino acids and with a DH of 33, of only 3 amino acids.

The antibiotic peptides of this invention may be applied to surfaces of plants or animals or added to foods to reduce bacterial count. Because the rate of kill of the peptide does not change significantly over a broad range of temperatures, the product to which the peptide is applied may be refrigerated without significantly reducing the rate of kill. This makes these antibiotic peptides especially useful for foods that are stored under refrigeration and for foods that are processed at room temperature and then transferred to an area under refrigeration. The peptide may be applied to any fruit or vegetable before, during, or after harvest to lower the bacteria levels, typically by 90 to 99%. While the kill rates fall off with fungi, an overnight treatment will still give a kill of 2 logs. As to bacteria, a 2 log kills occurs within hours even when the concentration of the peptide is as low as 3%. Fruits and vegetables that have been allowed to either vine ripen or tree ripen may be treated with the peptide to give the fruits or vegetables a longer life before spoiling. Removing most of the bacteria from the outside of fruits and vegetables also improves their taste.

A solution of the antibiotic peptide can be sprayed on the carcass of poultry or red meat animals to lower the counts on the outside of the carcass by 90 to 95% and the kill rate does not fall of when the carcass is refrigerated.

The antibiotic peptide may also be added to a medicine, ointment, or paste. The peptide can be used in human and veterinarian medicine to kill bacteria on or in a person or animal. For example, pets can be treated to lessen the bacteria counts in ears, nose, and under the coats. The peptide can also be used in shampoos, eye washes, nose drops, dentifrices, toothpaste, and other substances where antibiotic properties are desirable.

The peptides of this invention have no known safety problems at any level. It can be used in natural foods marketing and preservation since it is a natural food and can be used at any strength in any amount and at any point in the life of the natural food. It can be added to a cold drink for athletes and the drink can be packaged via a cold process (i.e., no sterilization). It can be added to food and drinks both for its antibiotic properties and also for its protein nutritive properties by substituting the peptides for intact protein in the food or drink. The following examples further illustrate this invention:

### EXAMPLES

Methods and Inoculums

In order to evaluate the antibiotic breadth of the antibiotic peptides of this invention a method of analysis and a suitable set of inoculums to test the peptides against were needed. Bacteria counts, yeast, and mold determinations and special bacterial counts for the coliform group portion of the bacteria colony were used for the inoculums. Standard methods agar were used for total count, potato starch dextrose agar for yeast and mold determinations, and Violet red bile agar for coliform determinations. Standard inoculums were used for the test work in order to follow the death curves of the bacteria, yeast, and mold. A 2 log death curve change was considered to be indicative of sufficient antibiotic activity to be considered a true antibiotic.

Fleishman's yeast preparation, sold in the grocery stores for making home made breads and wine, was used for a standard yeast preparation. A standard mold inoculum for mold determinations was prepared. Mold that was growing on fresh strawberries was the source of the mold. For a moderately contaminated bacterial colony, a commercial product (Proliant Peptide Product) that was close to spoiling before drying was used. The source of this product was from a cheese plant and the preparation was a WPC peptide product that had gotten away from them bacteriologically. (Proliant is a large cheese manufacturer. Several years ago Proliant was trying to make some whey peptides. They made samples of the product available in 2 quart plastic bottles marked “Proliant hydrolisate” for potential customers to evaluate. This product, “PPP” for Proliant Peptide Product, was assayed and found to smell and taste bad. It had a high bacteria count and the DH was 20%. It contained 1.5 million bacteria per gm.)

The following is a description of the two main inoculums used in almost all of the experiments:

Proliant Peptide Product (PPP)

This preparation had a count of 1.5 million. (log 6). When this inoculum was used at the 1 0 percent level, it added sufficient bacteria to raise the count to 1 50,000 or log 5. And when used at the 1% level the added bacteria had a log 4 count.

A locally purchased ground meat product was used for the highly contaminated inoculum. The ground meat product was stored under refrigeration for two weeks. A homogenate of this product was prepared and the connective tissue was screened out. This meat mix was standardized for starting count, both total plate count (tpc), coliforms, and molds. Typically, the tpc was log 8 and the coliform and fungal counts were log 5. Since in the Sawhill patent the WPC enzymatic digestion ended up at a pH of 4, a pH of 4 was used in this work as a starting point until the optimum pH could be found. The inoculums were also used at the 1 0% level and the WPC antibiotic at the 1 0 percent level in the first portions of the experimental work. Since these potential variables were the conditions at the end of the preparation of non-antigenic peptides of the Sawhill patent, it was a good point to start testing the various variables in the process of this invention on the antibiotic properties of a Formula 15 product.

It was found that DH values higher than about 1 5% do not further increase the antibiotic properties of the peptides. A longer period of digestion will increase the DH percent, but the antibiotic properties of the substrate remain about the same. High antibiotic peptide properties and high non-antigenic properties can be present in the same preparation. Conversely, the antibiotic properties can be present in the finished product without the presence of the non-antigenic factor. There are no problems with taste in either a 24 hour digestion or with a 48 hour digestion. There are no problems with taste when the concentration of the protein in the substrate is 1 0, 20, or 30% or any in between percentage. The finished product from the three different dilutions and the different times of digestion are almost identical in taste to the taste of the starting wpc and this has been verified by professional taste testing.

History of the Experiments with Key Experiments Discussed

As reported in Example 5 of the Sawhill patent, there was no problem with the enzymatic solutions spoiling during their 48 hour preparation. The tpc reported in Example 5 of the Sawhill patent showed that the bacteria levels were under control.

An experiment was performed (Example 1) in which a 200 mls of an aqueous solution that contained 10% WPC was split into two equal parts. The first portion was digested with 0.1%fungal “Proteinase 500,000” (Genencor) and the control portion contained no enzyme. Both of the samples were placed in a 105° F. forced air oven and digested for 48 hours. Each was agitated and the starting pH was 6.2. Every 6 hours samples were taken and the level of bacteria was determined by performing a total plate count. After 24 hours, the enzyme treatment sample had a second portion of enzyme added. At the end of the second 24 hour step, the total plate count was determined on both samples and the samples were tasted and smelled. The untreated sample was close to spoiling and smelled bad and tasted bad. The enzyme-treated sample had a pH of 4.2 and the untreated had a pH of 7. The enzymatic sample had a count of log 2 (100-1000). The control had a count of log 8 (100,000,000-1,000,000,000). The six hour bacteria count data points were graphed and it appeared that the antibiotic activity kicked in after 12 hours and was complete at 18 hours. This coincided with a DH of 15%

15 Formula Versus 25 Formula

In the key experiment of the series (Example 7), a two step experiment was performed that produced two products. The experiment was performed as described in Sawhill to produce non-antigenic peptides and had a DH% of over 25 at the end of the 48 hours; all of the criteria of the Sawhill patent were met. Since the antibiotic properties seem to appear at the end of the first day, a portion of the experimental solution was removed at the end of the first 24 hours. At this point, the solution had a DH% slightly over 15 and should have had all the antibiotic properties seen in previous experiment. This sample was marked “Formula 15” and the final sample at the end of 48 hours of enzymatic digestion was marked “Formula 25.” The Formula 15 sample was not non-antigenic but the Formula 25 sample was. For the evaluation of the two formulas for antibiotic activity, PPP inoculums were used with a log 5 level of bacterial inoculums for the evaluation. A 3 hour digestion of the inoculums was performed at 100° F. with stirring and each sample was counted. There was no difference between the two samples; both had the same amount of kill, two logs (99%) after 4 hours.

Scope of the Antibiotic Activity

Methods, Procedural and Ingredients

The following inoculums were used:

1. A dried cheese plant peptide product, which had a high tpc count (6).

2. Mold lifted from moldy strawberries, homogenized and diluted to a standard count.

3. An aged ground beef homogenate that had been stored for 2 weeks refrigerated to generate high count was evaluated and found to have a tpc of log 8 and a coliform count of log 5; the mold count was also log 5.

4. Fleishman's Yeast for bread making and wine inoculums, was used for yeast determinations as the substrate. The yeast product was homogenized with water and diluted until the yeast count was in the right range.

Media

All of the media was purchased fresh from the VWR Company and the protocol for these Medias in the Disco Manual were followed. The pipettes and Petri dishes were purchased sterile and the dilution waters were sterilized at 250° F. The tpc media and the potato starch media were made up and sterilized in the lab at 121° C. (250° F.) for one half an hour in a high pressure sterilizer. The sterilized media was used up within 3 days. The violet red bile agar was made up fresh just before use according to the Disco procedure, cooled, and the coliform plates poured at a temperature of 120° F.

A Kill of 2 Logs

To have a product that would be considered to be an antibiotic, the product should lower the level of bacteria 2 logs (99%). The same is also true for yeasts, molds, and fungi.

pH Effect on Bacteria

A test analytical series was run with the PPP inoculum at the 10% level and the antibiotic level at 10%. A series of pH's were tested for the degree of kill. The pH's were 3.5, 4.0, 4.5, 5.0, and 5.5. The temperature was 100° F., the samples were agitated during the 3 hour digestion period. The 3.5 and 4.0 sample had a 2 log kill . The other pH's had less than a log 2 kill. The pH 5 sample was about half of the pH 4 kill and pH 5.5 was practically zero

Effect of Stirring

In the first few series of experiments (Examples 1 to 16), the antibiotic test samples were stirred and the tests were performed at 100° F. Later, a couple of experiments were run that showed that stirring and the 100° F. temperature were unnecessary, so the antibiotic test solutions were no longer agitated during the several hours of the test.

Effect of Changing the Concentration of the Substrate in the Enzymatic Step During the Enzymatic Digestion of the WPC for the Production of Whey Antibiotic Peptides

Most of the experiments were performed at 20 to 25% solids with the standard amount of enzyme and 105 to 110° F. as the temperature during the digestion. One set of experiments (Example 9) was performed with a very low protein level in the WPC. This product made a good antibiotic and the conditions necessary were worked out to make the experiment a success. Two different WPCs were digested at the same time to evaluate the possibility of making antibiotic peptides when the concentrations of the WPC was around 30%. One of the suppliers of the starting materials was Leprimo's and the substrate WPC was 80% WPC. The second supplier was First District, a cheese manufacturer, who sold 34% protein WPC. The 34% WPC's pH was adjusted to 5 before the enzymatic was started. The enzyme level was 0.1% in both and the DH and pH of each were followed for 24 hours during the digestion. After the enzymatic was completed, both had a DH of more than 15% and each had antibiotic activity of log 2 over a 4 hour antibiotic assay using the PPP inoculums at the 10% level.

Time to Reach 2 Logs

When the conditions are optimum, such as pH, concentration of antibiotic, etc., tpc will reach 2 logs within 4 hours. It will reach one log with in 1 hour. For coliforms, the death curves are sharper and the one log death curve is reached in 30 to 45 minutes, 2 logs within 1.5 hours, and 3 logs within 3 hours. Yeast and mold death curves take longer. Within 3 to 4 hours neither yeast nor mold has reached 1 log but definitely show 50 to 80% kill. Overnight determinations on mold in the meat inoculums showed 2 logs after the extended time period. There is an indication that the mold optimum pH is 5 rather than 4, which is the optimum for bacterial antibiotic activity.

Temperature Effect on 2 Log Kills

At the start of experimental work the samples were stirred and the antibiotic death curves on the samples were performed at 100° F. Later, it was found that the antibiotic death curves are not influenced by either stirring or temperature. The death curves are the same for 35° F., 70° F., and 100° F., with and without stirring.

Coliforms

After most of the parameters governing the death curves of the antibiotic digestion using the different inoculums had been determined, the effect on just one portion of this large group of bacteria present in the meat inoculums, the death curves for the coliforms, was determined. Meat inoculums that had a log 5 coliform level of coliforms were used and experiments were performed using time and degree of the kill as the criteria. It was found that the antibiotic peptide solution at 10% will kill 4 logs of the coliform bacteria within a few hours time span. Since the experiments were started with log 5 inoculums, getting a better look at the curves would mean getting a higher degree of coliform contamination. As it is, the death curves were 99.99% effective (4 logs)

Yeast and Molds Pure and in a Mix

The first exploratory work performed in the laboratory utilized two different inoculums for evaluation. The yeast inoculum consisted of a pure yeast culture and the mold inoculum was a mold that had formed a colony on some strawberries. Since the antibiotic properties of the whey peptides had an optimum pH of 4 on bacteria and was more or less complete at that pH, those conditions were used for the yeast and mold determinations. All three curves were run on different inoculums. (tpc, coliforms, and fungals) simultaneously. There was a substantial amount of mold present in the aged ground beef inoculum (log 5). Some of these curves were run overnight, which showed that the mold death curves are also 2 logs, but it just takes longer for the antibiotic peptides to kill mold.

Log 2 Curves with tpc, Coliforms, and Mold

A decided difference was found between in the kill rates of the two bacteria types and mold. The kill rate is highest for the coliform group, next highest for the tpc group, and least high for the molds. Yeasts and molds take up to 12 hours, tpc takes up to 4 hours, and the fastest is the coliform group at 2 hours.

Testing the Antibiotic on a Chicken Leg

Using two 100 gm drumsticks from fresh killed chicken, several small experiments were run to see if tests on them could be evaluated. The drumsticks were cleaned thoroughly to eliminate most of their bacteria colonies. Each drumstick was dipped into the high count meat inoculum and was slightly dried by leaving both in a forced air oven at 100° F. for a few minutes. One drumstick was placed in a beaker and allowed to stand in the lab for 3 hours. The second drumstick was dipped in a 10% antibiotic solution, drained, and placed in a beaker and also allowed to stand for 3 hours. Each was then scrubbed with 50 mls of sterile water using a sterile cotton swab. The swab and the water wash were mixed thoroughly, 1 ml aliquots taken, and the tpc and coliform counts run. The starting inoculums bacteria levels were determined and the amount of sorption of the inoculums was calculated. The balance of applied bacteria and the amount collected from the control was good, thus making it possible to do a material balance. A 90% kill rate was achieved for the added bacteria both tpc and coliforms.

Effect of Concentration of Antibiotic on Kill Rates

In the starting work, the evaluations were performed at pH of 4; the concentration of the substrate was 10%, which meant that 8% of the test system was the antibiotic peptide. A series in which the amount of inoculum was kept constant and the amount of antibiotic peptide was varied was prepared. The pH was 4 and the concentration of the inoculum was 10%. The concentration of the “WPC 80” antibiotic peptide was from 0 to 10%. A 4 hr digestion at 70° F. was performed and the final products assayed for total plate count. The difference (delta kill rate in logs) was determined and compared for each of the concentrations of the peptide. The 8 to 10% peptide was a log 2 kill that was achieved fairly quickly. The group of 4% to 8% also had a good kill rate, but was slower. The 2 to 4% had bacteriostatic properties with some sign of kill of the bacteria in the inoculum.

When Do the Antibiotic Properties Show in WPC Hydrolysis?

Observations and comparison assays indicated that antibiotic properties appear in full when the WPC digestion reaches a DH of 15%. There may be an appearance earlier, but going to a 15% DH seems to insure that the reaction has gone to completion. Going to a higher DH neither increases nor decreases the antibiotic properties.

The appearance of the antibiotic peptide comes sometime during the first 24 hour digestion of whey peptides with the fungal proteinase. A 48 hour digestion with two enzyme increments indicated that the antibiotic properties begin to appear at the end of 12 hours, when the DH percent approaches 15. A DH of 15% was therefore used as the endpoint in several experiments. In one experiment, a two step experiment was performed using the standard operating procedure (SOP) of the Sawhill patent and removing a portion of the substrate at the DH 15 endpoint, which came at the end of the first day of enzymatic digestion. Half of the substrate was removed and marked “15” and the other half went on to the final DH of 25. The two samples were compared for antibiotic properties, but no difference was found.

Time and Concentration of the Substrate

It is an advantage to be able to perform the digestion of the whey protein concentrate to produce antibiotic peptides within 24 hours with a one step digestion. In Sawhill, the digestion to produce the non-antigenic peptides required the enzymatic to be done in a dilute system to minimize steric hindrance of the whey proteins. To determine whether steric hindrance was a problem the concentration of the substrate was increased in several experiments. In the Sawhill patent, the concentration was 10% but the allowed claims were wide enough to include 20% substrate concentrations. In this work, concentrations of 20 to 35% were performed and it was possible to achieve a DH of 15% at the higher concentrations of substrate. Both high concentrations of peptide in “WPC 80%” and low concentrations of peptide in the “WPC 34%” were evaluated and found to be acceptable.

Since the SOP utilizes 0.1% enzyme, there is a substantial savings on the costs of the enzyme. It costs one dollar to treat a pound of “WPC 80” and to transform it into suitable non-antigenic peptides. Because only one increment of enzyme is used at an increased level of WPC in the substrate, the savings are considerable. Two different conditions can further change this cost in a beneficial way. First, the use of a single enzyme step at a substantially higher concentration of substrate reduces the costs of the enzyme used in the preparation of the peptide to only 20% of the costs in Sawhill. And second, in Sawhill the final product is so dilute it has to be concentrated to remove some of the water, which is not required in this invention. Due to the susceptibility to denaturation, this concentration step is also expensive and must be performed under vacuum. These two changes in the process from a formula 25 to a formula 15 results in substantial savings of production costs.

Taste Evaluations

No difference in taste of the starting whey protein, the 15 formula products and the 25 formula product could be detected. Each of the potential starting WPCs had its own taste and this taste carried though the process. The 34% product has a stronger taste of salt and a sweeter taste of sugar than the 80% protein formulas. The contaminants in the starting WPC were lactose and ash. Since there are substantial differences in their levels in these two products, this results in differences in the final taste of the peptides made from them, but both products are acceptable and have no off odor or taste.

The DH 15 formulas were tasted by a knowledgeable taste tester in this work and were found to not change, or to change only slightly, in taste from the starting WPC taste. This also held true when the WPC was changed into the 25 formula.

### Example 1A

### Enzymatic Digestion of “WPC 80” Against Control

The purpose of this experiment was to examine the bacteria counts of an untreated control versus an enzymatically treated WPC in order to determine when the antibiotic properties appear.

Experimental

1. Made up 200 mls of a 10% solution of LePrimo's “WPC 80.”

2. Split the solution into two equal parts and added 0.1% of “Proteinase 500,000” to one of them.

3. Checked the pH and DH periodically throughout the experiment.

4. Placed both samples on magnetic stirrers inside a forced air oven at 105° F.

5. All samples were put up for bacteria counts. (tpc is reported in logs since the differences are of such a magnitude as to be not graph able.)

6. At 24 hours, a second increment of enzyme was added to the test system.

| | | | | DH of | |
|---|---|---|---|---|---|
| | Time | DH of | pH | experiment | pH of |
| Sample | (hrs) | control (%) | of control | (%) | experiment |
| 1 | 0 | 4 | 6.2 | 4 | 6.2 |
| 2 | 4 | 6 | 6.0 | 7 | 6 |
| 3 | 8 | 6 | 5.9 | 11 | 5.5 |
| 4 | 14 | 6 | 6.3 | 16 | 4.7 |
| 5 | 24 | 7 | 6.7 | 19 | 4.2 |
| 6 | 36 | 8 | 7.0 | 24 | 4.2 |
| 7 | 48 | 8 | 7.5 | 28 | 4.4 |

The results are given in the following graph:

Graph 1

Log of Bacteria Counts

[image]

### Bacterial Stability of a 25 Formula Made from Proliant WPC 80

Since there were problems with sterility, a new sterilizer was purchased and the media and dilution waters were freshly sterilized. A 25 Formula with “Proliant WPC 80” was prepared and a curve was run on DH%; samples were prepared for count. A control was run to see what the count was on a 48 hour sample of WPC without enzyme.

Experimental

Made up 400 mls of 10% and split into two equal parts. One was used as a control.

To the second one 200 mgs of “Proteinase 500” dissolved in 20 mls of water was added.

Placed both on stirrers in the forced air oven at 100° F.

Periodically the pH was checked, the DH determined, and the appropriate bacteria counts w ere run for each of th e two samples.

The enzymatic and control samples were run for 48 hours.

| | | | | | | Log of |
|---|---|---|---|---|---|---|
| Sample | Type | pH | DH (%) | Time (hrs) | Bacteria | bacteria |
| 1 | Control | 6.2 | 4 | 0 | 2,200 | 3.2 |
| 2 | Control | 6.2 | | 4 | 22,000 | 4.2 |
| | Enzyme | 6.0 | | | 25,000 | 4.3 |
| | Enzyme | 5.5 | | 8 | 3000 | 3.3 |
| | Control | 5.9 | | | 15M | 7.1 |
| 4 | Control | 6.3 | | 14 | 20M | 7.2 |
| | Enzyme | 4.7 | | 14 | 100 | 2.0 |
| 5 | Enzyme | 4.2 | | | 80 | 1.8 |
| | Control | 6.7 | | 24 | 300M | 8.3 |
| 6 | Enzyme | 4.2 | | | 29 | 1.3 |
| | Control | 7.0 | | 36 | 500M | 8.9 |
| 7 | Enzyme | 4.4 | | | 10 | 1.0 |
| | Control | 7.5 | | 48 | 700M | 8.9 |

The purpose of this experiment was to find out what happens to a control that has a count of close to a billion when treated in one of two ways. First, what happens when a peptide formula is added to the almost spoiled WPC solution from this experiment? And second, what happens when the enzyme is added to the mix and a 24 hour digestion is performed?

Procedure

1. Split the 200 ml's of the control in half.

2. The first half treated with 10% added 3×3 and pH is adjusted down to 4.3

3. The second half was committed to an enzymatic then 0.1% “Protease 500” (100 mgs) was added. The enzyme had been dissolved in 10 mls of water before the addition.

4. The two 100 ml preps were put up in the constant temperature forced air oven and stirred for 24 hours.

5. Samples were taken at 12 hours and 24 hours. Bacteria run.

### Example 2

### Preparing a Suitable Bacteria Inoculum

A standard was needed to use in experiments in order to control the load on the system.

Experimental

1. Made up 100 mls of a 1% and a 100 ml of a 10% solution of “PPP.”

2 Set up to stir at 100° F. for 12 hours.

3. Removed samples periodically and counted.

4. In the tables, “T” is thousand and “M” is million.

Results

| Sample | Time (hrs) | 19% Count | 1% count |
|---|---|---|---|
| 1 | 0 | 65 T | 25 T |
| 2 | 4 | 7 M | 800 T |
| 3 | 8 | 250 M | 100 M |
| 4 | 12 | 300 M | 250 M |

Usage

1. For a low starting count, samples of either 10% or 1% can be used.

2. For a medium count 1% can be used at two hours.

3. For a higher count a 4 hr sample of 1% can be used.

This is chicken drumstick data.

| Sample | TPC | Coliform (/ml) | Log of tpc | Log of coliforms |
|---|---|---|---|---|
| Inoculum | 330 M | 800 T | 8.3 | 5.9 |
| 50 ml Cont. | 300 M | 700 T | 7.1 | 5.5 |
| Wash | | | | |
| Amt of bacteria | 300 M | 700 T | 5.9 | 5.7 |
| in wash of | | | | |
| control | | | | |
| Test 50 ml | 500 T | 50 T | 5.7 | 4.6 |
| wash of treated | | | | |

1. The actual amounts of bacteria agreed fairly well between the theoretical and actual. The bacteria were killed at over 99% and the coliforms at over 90% also.

[image]

### Example 3

### Optimum Level of Peptides for Antibiotic Activity

Discussion

This experiment was to determine the effect of different concentrations of the anti-biotic activity on standard inoculums. A mixture of two solutions was used to check most of the possibilities. The temperature was 40° C. with mixing for 4 hours.

Experimental

1. Make up a 20% solution of the 3×3 antibiotic peptide at pH 4.5.

2. Make up a 10% solution of Proliant PP pH 4.5.

3. Using the formulas appearing in Table #1make up 15 ml samples and place them on a shaker in 15 ml centrifuge tubes.

4. Mix for four hours and then count.

Table #1

Proportions in the Experiment

TABLE #2

| | Antibiotic | | | | |
|---|---|---|---|---|---|
| | peptide | | | | |
| | solution | | | Inoculum | Water |
| Sample | (mls) | Peptide (g) | Peptide (%) | (mls) | (mls) |
| 1 | 10 | 8 | 10.6 | 5 | 0 |
| 2 | 9 | 7 | 9.4 | 5 | 1 |
| 3 | 8 | 6.4 | 8.5 | 5 | 2 |
| 4 | 7 | 5.6 | 7.1 | 5 | 3 |
| 5 | 6 | 4.8 | 6.3 | 5 | 4 |
| 6 | 5 | 4.0 | 5.3 | 5 | 5 |
| 7 | 4 | 3.2 | 4.2 | 5 | 6 |
| 8 | 3 | 2.4 | 3.8 | 5 | 7 |
| 9 | 2 | 1.6 | 2.1 | 5 | 8 |
| 10 | 1 | 0.8 | 1.1 | 5 | 9 |

Bacteria Counts, Log of Count and Delta Log Data

| Sample | Count | Log | Delta Log |
|---|---|---|---|
| 1 | 20 | 1.2 | 3.8 |
| 2 | 50 | 1.7 | 3.2 |
| 3 | 2000 | 3.3 | 1.6 |
| 4 | 2000 | 3.3 | 1.6 |
| 5 | 3000 | 3.5 | 1.5 |
| 6 | 2000 | 3.3 | 1.5 |
| 7 | 16,000 | 4.2 | 0.65 |
| 8 | 15,000 | 4.2 | 0.65 |
| 9 | 800,000 | 5.9 | −1.0 |

Results

1. The two highest levels 10&9% are the best and can be considered to kill 99.95% of the bacteria present

2. The 4 to 8 percent level kills 95% of the bacteria colony

3. The 2 & 3 percent level kills 50% of the bacteria

4. The 2% level does not kill anything. Intact the colony grew from 100,000 to 180,000 per ml.

[image]

### Example 4

### Effect of pH on Death Curves

Previous work indicated a pH of 3.5 to 4.5 was acceptable. This experiment was performed to determine the effect of pH over a large range.

Experimental

20% solutions of inoculums were mixed with 20% 3×3 at the 16 ml size and a pH curve was run. The samples were held at 100° F. with mixing. The length of time was 4 hours for the digestion.

Prepared 100 ml of 20% solution of 3×3.

Prepared 100 liters of a 20% solution of Proliant peptides (inoculums).

Mixed the two liquids and ran a 0 time tpc.

Lowered the pH with sulfuric acid and took periodic samples for testing purposes.

Placed samples in 15 ml centrifuge tubes and placed on shaker at 100° F.

Mixed for four hours and then ran bact T on each.

TABLE #1
pH and tpc Results

| Sample | pH | Bacteria Count | Log of Count | Delta Log |
|---|---|---|---|---|
| 1 | 7.2 | 500,000 | 5.1 | 0.9 |
| 2 | 6.6 | 1,200.000 | 6.1 | 0 |
| 3 | 6.0 | 800,000 | 5.9 | 0 |
| 4 | 5.2 | 270,000 | 5.4 | 0/6 |
| 5 | 4.7 | 12,000 | 4.1 | 1.9 |
| 6 | 4.2 | 2,500 | 3.4 | 2.6 |
| 7 | 3.8 | 250 | 2.4 | 3.6 |
| 8 | 3.5 | 160 | 2.2 | 3.8 |
| 9 | 3.0 | 1400 | 3.1 | 2.9 |

The starting counts before digestion were 500,000/mi (log 5.8)

Results

The pH's were previously set too high for testing.

The optimum pH seems to be from 3.5 to 4.0.

The slight rise in bacteria count at a pH 3 is surprising.

The bacteriostatic properties kick in around 5.2 and bacteriocidal from 4.7 down to 3.

| Series | pH | Log Count | Delta log |
|---|---|---|---|
| 9 | 3 | 2.9 | 2.9 |
| 8 | 3.5 | 2.2 | 3.8 |
| 7 | 3.8 | 2.4 | 3.6 |
| 6 | 4.2 | 3.4 | 2.6 |
| 5 | 4.7 | 4.1 | 1.9 |
| 4 | 5.2 | 5.4 | 0.6 |
| 3 | 6 | 5.9 | 0 |
| 2 | 7.2 | 5.1 | 0 |
| 1 | 8.6 | 6.1 | 0 |

[image]

### Example 5

### Time Required to Produce Bacteriostatic and Bacteriocidal Conditions

The purpose of this experiment was to determine the optimum digestion time. The peptide solution was 10% of an 80% solution at pH 4.0 and the temperature was 100° F. with stirring.

Procedure

1. Place 80 mls of water in a forced air oven and bring the temperature up to 100° F.

2. Add 10 gms of antibiotic peptide & 110 gms of “PPP” (inoculums).

3. Adjust pH to 4.0 with sulfuric acid.

4. Take 0 time sample for tpc.

5. Take 30 min, 1 hr, and 2 hr samples for tpc.

6. Take additional samples for tpc at 3, 4, and 7 hours.

| Sample | Time (hrs) | Count | Log of count | Delta log | % Killed |
|---|---|---|---|---|---|
| 1 | 0 | 2 M | 6.3 | 0 | None |
| 2 | ½ | 400 T | 5.6 | 0.7 | 80 |
| 3 | 1 | 300 | 5.5 | 0.8 | 85 |
| 4 | 2 | 200 | 5.3 | 1.0 | 90 |
| 5 | 3 | 200 | 5.3 | 1.0 | 9 |
| 6 | 4 | 70 | 4.8 | 1.5 | 94 |
| 7 | 7 | 44 | 4.6 | 1.8 | 98 |

It appears that the death curve continues on after 10 hrs.

| Time (increment) | | Time (hours) | Incremental % kill |
|---|---|---|---|
| 0-½ | hr | 0.5 | 80% |
| ½-1 | hr | 1 | 25% |
| 1-2 | hr | 2 | 33% |
| 2-3 | hr | 3 | 25% |
| 3-4 | hr | 4 | 25% |
| 4-7 | hr | 7 | 10% |
| 7-10 | hr | 10 | 10% |

[image]

### Example 6

### Death Curves at 2 Temperatures and 2 Concentrations

The purpose of this experiment was to determine the level of the bacteria in the inoculums (“PPP”) and to plot the death curves over a longer period of time. Inoculums of PPP at 1% and 19% and temperatures of 70 and 100° F. were used.

Procedure

1. Made 140 m/s of a 10% solution of PPP. Adjusted pH to 4 with sulfuric acid.

2. Removed 6 mls and diluted to 60 mls.

3. Made up two 60 ml samples from the 140 ml of starting material.

4. Put one 10% sample in a forced air oven with agitation using a magnetic stirrer. The other sample was kept at ambient temperature in the laboratory.

5. The pH of all three samples was adjusted to 4 with sulfuric acid.

6. Samples were taken at 0 time and the tpc was run.

7. The samples were checked three more times, at 3 hours, 10 hours, and again at 24 hours.

Data

| | | | | Bacteria | Log of | Delta |
|---|---|---|---|---|---|---|
| Sample | Temp (° F.) | Time | Hours | count | count | log |
| 1 | 100 | 10 AM | 0 | 210,000 | 5.3 | 0 |
| | | 1 PM | 3 | 3,000 | 3.5 | 1.8 |
| | | 8 PM | 10 | 1500 | 3.2 | 2.1 |
| | | 9 AM | 23 | 400 | 2.6 | 2.7 |
| 2 | 70 | 10 AM | 0 | 210,000 | 5.3 | 0 |
| | | 1 PM | 3 | 7,000 | 3.8 | 1.5 |
| | | 8 PM | 10 | 230 | 2.5 | 2.8 |
| | | 9 AM | 23 | 100 | 2.0 | 3.3 |
| 3 | 100 | 10 AM | 0 | 15,000 | 4.15 | 0 |
| | | 1 PM | 3 | 1300 | 3.1 | 1.0 |
| | | 8 PM | 10 | 280 | 2.4 | 1.7 |
| | | 9 AM | 23 | 100 | 2.0 | 2.1 |

| Time | 100 F./10% | 70 F./10% | 100 F./1% |
|---|---|---|---|
| 0 hrs | 0 | 0 | 0 |
| 3 hrs | 1.8 | 1 | 1.5 |
| 10 hrs | 2.1 | 1.7 | 2.8 |
| 20 hrs | 2.7 | 2.1 | 3.3 |

[image]

### Example 7

### Preparation of Formulas 15 and 25 for Test

This experiment was to determine whether there was a difference in the antibiotic qualities of a Formula 15 and a Formula 25 compared to an antibiotic pilot plant production (i.e., peptide prepared according to the Sawhill patent using Enamor's Proteinase 500,000 in both digestions.) A Formula 25 experiment was performed on the new substrate and half was removed at the end of the first day, i.e., the Formula 15, and put into the refrigerator and then continued on with making the Formula 25. When the Formula 25 was finished it was stored along with the Formula 15 in the refrigerator and then the two were compared along with a 3×3 sample to determine their antibiotic qualities.

Procedure

1. Dissolved 30 gms of WPC (“LePrimo 80”), in 270 mls of water.

2. Added 300 mgs of enzyme (Genencor's “Proteinase 500,000”).

3. After 24 hours, removed 150 mls, adjusted the pH to 4, and stored the digest at 40° F.

4. Added 150 mgs of enzyme to the remaining digest and continued the digestion for an additional 24 hours.

Preliminary Testing Data

1 The Formula 15 had a DH of 14 and a pH of 4.3.

2. The Formula 25 had a DH of 27 and a pH of 4.9.

Both digests were acidified and stored overnight in the refrigerator and then assayed for death curves on PPP art the 1% level.

Procedure

1. 50 mls of each digest was placed in a forced air oven at 100° F. and stirred. PPP was added (1%, 500 mgs) and dissolved.

2. One ml samples were removed at the appropriate time and assayed for bacteria levels.

Data

| | | Formula 15 | | | | Formula 25 | | | |
|---|---|---|---|---|---|---|---|---|---|
| | | | | Delta | Log | | | Delta | Log |
| Step | Time (hrs) | Count | Log | log | (%) | Count | Log | log | (%) |
| Digest + PP | 0 | 15,000 | 4.3 | 0 | 0 | 15,000 | 4.3 | 0 | 0 |
| 15 min | ¼ | 6,000 | 3.8 | 0.4 | 60 | 6,000 | 3.8 | 0.4 | 60 |
| @ 100° F. | | | | | | | | | |
| 30 min | ½ | 5,000 | 3.7 | 0.5 | 65 | 4,000 | 3.4 | 0.9 | 70 |
| 2 hr | 2 | 500 | 2.7 | 1.6 | 97 | 400 | 2.6 | 1.7 | 97 |
| 20 | 20 | 50 | 1.7 | 2.6 | 99 | 10 | 1.0 | 3.3 | 99.9 |

Results

1. There did not seem to be any difference between the two preparations. The one day digestion appeared to be the same as the two day digestion (15 is equal to 25).

2. The death curves were not as steep in the beginning as some were in previous experiments. (See graphs in Example 8 below for further details.)

### Example 8

### Evaluation of Three Pilot Scale Production Runs for Antibiotic Activity

Three different pilot runs have been used as the standard in the evaluation of different aspects of the 15 and 25 formulas of antibiotic peptides. This experiment was performed in order to determine whether all three of the pilot runs were equivalent, so three different peptide products were run against each other to see if there were any differences. New samples were taken from the parent materials in storage and each was tested against the PPP inoculum to see if they had about the same kill power (1.5 to 1.8 logs) on the PPP inoculum over 6 hours. The three pilot scale preparations were 3×3, 3×2, and 2×2.

Experimental

1. Prepared three identical samples of 100 mls each, containing 10 gms of the peptide and 10 gms of PP inoculums.

2. The pH was adjusted to 4 with sulfuric acid.

3. The samples were stirred well, covered with aluminum foil, and set out on a bench top for 6 hours.

5. Samples were taken at 0 hrs, 3 hrs, and 6 hrs.

6. Total plate count was determined for each and the data appears in table #1

| Sample | Log of 0 hr tpc | Log of 3 hr tpc | Log of 6 hr tpc |
|---|---|---|---|
| 3 × 3 | 5.0 | 3.0 | 3.3 |
| 3 × 2 | 5.0 | 3.2 | No sample |
| 2 × 2 | 5.0 | 2.8 | 2.8 |

Results

1. All three preps made in the pilot plant two years ago were still active as antibiotic peptides but had slightly less killing power than the 15 Formula.

2. There were very few differences between the three samples in death curves for total plate count.

Log of Kill

| Time | 3 × 3 | 3 × 2 | 2 × 2 |
|---|---|---|---|
| 0 hrs | 5 | 5 | 5 |
| 3 hrs | 3.1 | 3.2 | 2.9 |
| 6 hrs | 3.5 | | 2.9 |

[image]

### Example 9

### Evaluation of New WPC 33%

Previous work showed that the protein in this dilute product would respond to the enzyme. The problem with this material was the dilute status of the WPC. To make an 8% solution of the WPC protein would require making the system 24 wt % solids. This is acceptable for the 25 wt % formula but there is no way that twice this amount can be made into a solution for the 15wt % formula.

Experimental

1. Dissolved 100 gms of the WPC in 200 mls of water and determined the DH before enzyme.

2. Unfortunately, this material has a very high blank. Normally, there is a 2 ml 4% DH in the starting material. In this case, the starting pH was in the mid 5's instead of 6.2. It required 10 mls of 0.1 N NaOH to adjust the pH to 8. This is a very high blank

3. Added 300 mgs of enzyme and placed the sample in a forced air oven overnight. The DH titration required 22 mls. Allowing for the 10 ml blank, this equates to a 12 ml DH or a DH increase of 24.

4. Checked the DH a second time at 24 hours. 24 mls of base for a DH of 28

5. Removed 50 mls for future testing.

6. Added second increment of enzyme for an additional overnight digestion.

7. The final DH was 34% when corrected for the large blank.

| | | Volume | |
|---|---|---|---|
| Sample | Time (hrs) | of base (ml) | Corrected DH (%) |
| 1 | 0 | 19 | 0 |
| 2 | 12 | 24 | 10 |
| 3 | 24 | 30 | 22 |
| 4 | 28 | 47 | 28 |
| 4 | 48 | 51 | 32 |

Evaluation of Finished Products' Bacteria Levels after the 48 Hour Digestion

1. Put up finished product for count.

2. Ran finished product against 10% PPP to see if it had bacteriostatic properties.

Results of tcp

1. The final count on the peptides was <10/gm.

2. The starting solution was made up to contain 10% antibiotics from this experiment and 10% PPP for a starting count of 150,000 or a log of 5.1. The final count, after 4 hours of digestion, was a surprisingly low <100 bacteria per ml. This equates to a kill of delta log 3.1.

3. The 3 hour count on the 10% inoculums+10% PPP was 40/gm.

| | log | |
|---|---|---|
| Time | count | delta log |
| 0 hr | 5.2 | 0 |
| 4 hr | 1 | 4.2 |

[image]

Conclusions

1. First District's 34% WPC was one of the better substrates because it gave a good DH and was a superior antibiotic.

2. The taste was acceptable.

### Example 10

### Coliform Evaluations of Time

An attempt was made to use standard inoculums in the experimental work for testing several unknowns, such as time of death, pH, and concentrations.

A pound of ground beef was purchased and allowed to stand in a refrigerator for several days. Then the package was autolized overnight at room temperature. Fifty grams were removed and homogenized with 10 volumes of water. Coliform levels were checked and the homogenate was stirred at a constant temperature of 100° F. The first homogenate had a count of 70,000 and the second had a slightly lower count. While this is satisfactory, the inoculum count can be lowered 2 or more logs during the evaluation of the antibiotic qualities of the peptide against coliforms a lowering from log 4 to a log 2.

Experimental

1. Took 3 fifty ml samples of the homogenate and placed one in the laboratory at 60° F.

2. The second homogenate was placed in a 100° F. forced air oven and was allowed to come up to temperature.

3. Five grams of antibiotic peptide was added to each and the pH was adjusted to 4 with sulfuric acid.

4. The two temperature digestions were started.

5. Samples were taken at 15 min, 30 min, 45 min, 1 hour, and 2 hours.

6. The samples were put up immediately for counts at the appropriate dilutions, starting with 10 to the third and 10 to the fourth, then making the samples higher in sample percentages (See following table for results.)

| Sample | Temp. (° F.) | Time (min) | Log | Delta log |
|---|---|---|---|---|
| 1 | 70 | 0 | 4.9 | 0 |
| 2 | 100 | 15 | 4.3 | 0.6 |
| | 60 | 15 | 4.3 | 0.6 |
| 3 | 100 | 30 | 4.3 | 0.6 |
| | 60 | 30 | 4.3 | 0.6 |
| | 100 | 45 | 4.2 | 0.7 |
| | 60 | 45 | 4.1 | 0.8 |
| | 100 | 60 | 3.6 | 1.3 |
| | 60 | 60 | 3.4 | 1.5 |
| | 100 | 120 | 1.0 | 3.9 |
| | 60 | 120 | 1.2 | 3.7 |

1 There seemed to be little difference between the two temperatures.

2. 90% of the coliforms were destroyed within one hour.

3. 99.9% of the coliforms were destroyed within 2 hours

[image]

### Example 11

### Death Curves of a Highly Contaminated Meat Sample, Both Coliforms and Total Count

The two different types of death curves (coliform and tpc) were followed individually. The tpc counts were followed using inoculums of “PPP” that was contaminated with 1.5 million tpc/gm. Ground beef was used to prepare a highly contaminated beef sample that had a high coliform count. In this work, the meat type sample was used as the inoculums and an antibiotic peptide were used as a peptide source. Three variables were checked, a pH of 4 versus a pH of 5, a temperature of 50° F. versus a temperature of 100° F., and stirred versus unstirred.

Procedure

1. Removed special stored beef sample that had been allowed to stand at room temperature for several hours and then at a refrigerated temperature for several days.

2. Ran tpc and coliform levels with violet red bile agar and the total count with TPC peptone agar.

3. The 200 ml sample was made at a 10% concentration with 20 gms of 3×3 peptides.

4. Adjusted the pH to 5 with sulfuric acid and removed 50 mls.

5. Adjusted remaining 150 mls to pH 4 with sulfuric and split into three parts.

6. The four samples were stored at 55° F. or at 100° F. for the incubation period.

7. Some samples were stirred and some were not. (See the following table for details.)

8. The four samples were assayed at 8 hrs, 24 hrs, and 48 hrs for coliforms and 0 times for tpc.

9. The following table gives the results:

| | | | Temp. | Time | Coliform | |
|---|---|---|---|---|---|---|
| Sample | Stirred? | pH | (° F.) | (hrs) | (/ml) | TPC (/ml) |
| 1 | Yes | 5 | 60 | 0 | 320,000 | 6,000,000 |
| | | | | 8 | 12,000 | |
| | | | | 24 | 10,000 | |
| 2 | No | 4 | 60 | 0 | 320,000 | 6,000,000 |
| | | | | 8 | 10 | |
| 3 | Yes | 4 | 60 | 0 | 320,000 | 6,000,000 |
| | | | | 8 | 4,000 | |
| | | | | 24 | 10 | |
| 4 | Yes | 4 | 100 | 0 | 320,000 | 6,000,000 |
| | | | | 8 | 2,000 | |
| | | | | 24 | <10 | |

There is no tpc data on Sample 2 at 24 hours because it had gone too low to be readable.

[image]

### Example 12

In a previous experiment it was found hat 60% of the yeast was killed in four hours and mold was killed 90%, also in 4 hours. The previous work was performed at a pH of 4 and the concentration of the antibiotic peptide was 10%. The solutions were stirred and the temperature was 60-70° F. The test was discontinued after 4 hours.

Procedure

Made up 100 ml 10% solution of an antibiotic peptide solution and adjusted the pH to 5.5.

Added 0.1 ml of mold inoculums.

Ran 0 time mold count.

Set up 2 series of test tubes to contain samples. One series to be shaken on the mechanical shaker and one series to be stored without shaking.

Removed 10 mls for tubes 1&2.

Adjusted pH to 5 and removed 10 mls for tubes 3 & 4.

Adjusted pH to 4.5 and removed 10 mls for tubes 5&6.

Adjusted pH to 4 and removed 10 mls for tubes 7&8.

Put up 10/2 and 10/3 dilutions for mold count.

The starting count was 200,000, log 5.3.

The following table gives the results:

| Sample | pH | Stirred? | Final count | Log | Delta log |
|---|---|---|---|---|---|
| 1 | 5.5 | Yes | 25,000 | 4.4 | 0.9 |
| 2 | 5.5 | No | 60,000 | 4.7 | 0.6 |
| 3 | 5 | Yes | 20,000 | 4.3 | 1.0 |
| 4 | 5 | No | 4,000 | 3.5 | 1.8 |
| 5 | 4.5 | Yes | 40,000 | 4.5 | 0.8 |
| 6 | 4.5 | No | 38,000 | 4.4 | 0.9 |
| 7 | 4 | Yes | 100,000 | 5.1 | 0.2 |
| 8 | 4 | No | 120,000 | 5.2 | 0.1 |

Results

The mold kill seems on average to be 90% between the pH of 4 and 5.

Stirring versus unstirred is undecided. There is certainly the chance unstirred runs are acceptable.

[image]

### Example 13

### Temperature Effect on the Death Curves of Bacteria With and Without Stirring

The effects of temperature and stirring on the kill curves of WPC peptides against standard inoculums were determined. Previously, experiments were run with stirring and at two temperatures. The temperatures were 100 and 70° F. There was no difference. In most cases, the experimental work had been done with stirred samples. Since experiments on stirred refrigerated samples could not be easily performed, three unstirred samples and one stirred sample were used to check on the effect of temperature and differences between stirred and unstirred. Three temperatures were used: incubator, ambient lab, and refrigerator temperature. A duplicate sample was run at 70° F. that was stirred on a magnetic stirrer.

Procedure

1. Made up a 300 ml solution that contained both 10% of 3×3 peptide and 10% “PPP”.

2. Adjusted pH to 4 with sulfuric acid.

3. Three 15 ml samples in capped centrifuge tubers were removed from the mixture of antibiotic peptide and PPP inoculum. Each was placed at different temperatures. One was placed in an incubator at 100° F., the second in the laboratory at 70° F., and the third in a refrigerator at 35° F. The samples were not mixed or agitated with the exception of the #3 sample, which was mixed at room temperature on a shaker.

4. After three hours, the samples were removed and were plated out at several dilutions. (100,1000, and 10,000 dilutions)

5. The starting count of the “PPP” was 1,800,000, making the starting count of the 10% solution of PPP 180,000 (log 5.13) in the 10% inoculum addition.

Results

| Temperature (° F.) | Stirred? | Count | Log of count | Delta log |
|---|---|---|---|---|
| 100 | No | 4,000 | 3.6 | 1.5 |
| 70 | No | 6,000 | 3.7 | 1.4 |
| 70 | Yes | 5,000 | 3.6 | 1.5 |
| 35 | No | 7,000 | 3.8 | 1.3 |

Results

1. There does not seem to be any major differences between stirred and unstirred and no major differences between the three temperatures.

[image]

### Example 14

### Evaluation of Kill Rates of a High Count Inoculums on Chicken Leg

Very high count inoculums were prepared from hamburger. The total count should be in the 100's of million, the coli form count should be in the 10 to the sixth range, and the yeast and mold counts should be in the thousands. Two chicken legs were cleaned with a commercial spray and a water wash, and then dipped into the inoculums. The legs were warmed to 100° F. for several minutes to fix the inoculums to the legs, and then incubated. The control had no wash but the other was dipped into a 10% solution of 3×3. The two legs were stored in the laboratory at room temperature for 3 hours and then each was scrubbed with 50 mls of distilled sterile water. The 50 ml solutions were counted and the amount of each of the bacterial colonies determined. The legs were again washed off with the cleaning spray and the cleaning solution counted a second leg will be used as a control.

Procedure

1. Purchased a tray of 10 drumsticks from fryer chickens.

2. Refrigerated them for future work.

3. Removed two drumsticks and cleaned them up with the vegetable spray that dissolves waxes

4. Washed with water and saved solution for bacteria counts.

5. Dipped both drumsticks in the high count inoculums and allowed to drip dry

6. Suspended the drumsticks for fifteen minutes in 100° F. forced air oven.

7. Marked one of the drumsticks the control and stored the drumstick in the lab at room temperature.

8. The second drumstick was dipped in a 10% solution of 3×3 at pH 4.0

9. The second drumstick was also allowed to stand in the lab for three hours.

10. Both drumsticks were washed clean of inoculum and bacteria with 50 ml of water and cotton swabs.

11. The water washes were counted for tpc, coliforms, yeast, and mold.

Solutions to Count

1. Inoculums.

2. 50 ml wash of the control drumstick.

3. 50 ml wash of the test drumstick.

Weight Relationships

1. Weight of chicken drumsticks was 100-105 gms.

2. Amount of inoculums absorbed by each drumstick was 1-1.05 gms.

3. Amount of antibiotic peptide solution coating the test drumstick was 1.1 gms or 1.1 mls. The amount was determined by weighing the drumsticks before and after.

4. Volume of 3 hr wash of the control and the volume of the 3 hr wash of the unknown drumstick was 50 mls.

Assays

1. TPC with tpc agar, coliform count with violet red bile agar.

2. Weight of each of the two drumsticks was 100 gms ±5 gms.

3. Amount of inoculums was 2 gms/mls.

4. Amount of 10% antibiotic peptide solution pH 4 on the test drumstick was also 2 mls.

5. The theoretical amount of total plate count and coli form count was calculated and then the actual amount of the count in the 50 ml wash water of each drumstick was measured.

The following table gives the results:

| Sample | TPC | Coliform (/ml) | Log of tpc | Log of coliforms |
|---|---|---|---|---|
| Inoculum | 330 M | 800 T | 8.3 | 5.9 |
| Theoretical | 330 M | 800 T | 8.3 | 5.9 |
| amount in 1 ml | | | | |
| 50 ml cont. | 300 M | 700 T | 7.1 | 5.5 |
| wash | | | | |
| Bacteria in | 300 M | 700 T | 5.9 | 5.7 |
| wash of control | | | | |

The percentage of kill over the control was 99.5% for the tpc and 93% for the coliforms. This means 99.5% of the added bacteria on the unknown drumstick were killed and 93% of the coliforms were also killed. The actual amounts of bacteria agreed fairly well between the theoretical and actual.

[image]

### Example 15

### Evaluation Over Time of an Antibiotic Peptide Against Highly Contaminated Inoculums

Very high count inoculums were prepared from hamburger that had a tpc count of log 8, a coli form count of log 5, and a yeast count was also a log 5.

Experimental

1. Removed 200 ml inoculums from refrigerator and performed a count.

2. Added 20 gms of a Formula 25 antibiotic peptide and adjusted the pH to 4 with sulfuric acid.

3. After 6 hours, repeated the count.

4. After an additional 6 hours, performed a third count

| | 6 hr | Kill | 12 hr | |
|---|---|---|---|---|
| Measurement | (delta log) | (%) | (delta log) | Kill (%) |
| TPC | 2.1 | 99 | 2.1 | 99 |
| Coliform count | 2.5 | 99.5 | 2.5 | 99.5 |
| Yeast & mold | 1.3 | 93 | 1.9 | 98 |

Results

1. Within 6 hrs the total count and coliform counts had dropped over 2 logs

2. Within 12 hours the yeast and mold counts had also dropped 2 logs.

What is claimed is:

1. A method of making an antibiotic peptide comprising hydrolyzing milk protein in a single step at a protein concentration of 21 to 35 wt % with a fungal protease to a DH of at least about 12%.

2. A method according to claim 1 wherein said milk protein is whey protein concentrate.

3. A method according to claim 1 wherein said milk protein is hydrolyzed to a log 2 bacteria kill.

4. A method according to claim 1 wherein about 50% of the product of said hydrolyzation is free amino acid and 2 to 4 amino acid chains and the remainder is longer peptides.

5. A method according to claim 1 wherein said protein concentration is about 25 to about 33 wt %.

6. A method according to claim 1 wherein said protease is made from a fungus selected from the group consisting of subtiliasin Carlsburg, Aspergillus orzae, and mixtures thereof.

7. A method according to claim 1 wherein said protein is hydrolyzed to a DH between 15 and 25%.

8. A method according to claim 1 wherein the concentration of said protease is about 0.01 to about 1%.

9. A method according to claim 1 wherein said hydrolyzation is monitored by high pressure liquid chromatography.

10. A method according to claim 1 wherein said whey protein concentrate is about 65 to about 80% protein.

11. A method according to claim 1 wherein said hydrolyzation is performed at about 80 to about 120° F.

12. A method according to claim 1 wherein said protein is hydrolyzed at a pH of about 3 to about 5.5.

13. A method according to claim 1 wherein said hydrolyzation is performed for less than 24 hours.

14. An antibiotic peptide having at least a log 2 kill of bacteria, made according to the method of claim 1.

15. A animal or plant surface coated with an antibiotic peptide according to claim 14.

16. A food, medicine, ointment, or paste containing an antibiotic peptide according to claim 14.

17. A method of making an antibiotic peptide having at least a log 2 kill of bacteria comprising hydrolyzing milk protein at a concentration of 25 to 35 wt % in a single step with a fungal protease at a temperature of about 80 to about 1200F and a pH of about 3 to about 5.5 to a DH of about 15 to about 25%.

18. An antibiotic peptide having at least a log 3 kill of bacteria, made according to the method of claim 17.

19. A method of making an antibiotic peptide comprising hydrolyzing whey protein concentrate at a concentration of 25 to 33 wt % in a single step for less than 24 hours with a fungal protease selected from the group consisting of subtiliasin Carlsburg, Aspergillus orzae, and mixtures thereof at a temperature of about 105 to about 110° F. and a pH of about 3.5 to about 4.5 to a DH of about 15 to about 25% and at least a log 3 kill of bacteria.

20. An antibiotic peptide made according to the method of claim 19.
